(19) United States
(12) Patent Application Publication
(10) Pub. No.: US 2010/0166683 A1
(43) Pub. Date: Jul. 1, 2010

(54) COMPOSITION OF EXTERNAL PREPARATION FOR UV-PROTECTION COMPRISING REHMANNIA GLUTINOSA EXTRACTS

(76) Inventors: Byung Ki Suh, Seoul (KR); Jung Seol Suh, Seoul (KR); Jung Gun Suh, Seoul (KR)

Correspondence Address: FROMMER LAWRENCE & HAUG, 745 FIFTH AVENUE- 10TH FL., NEW YORK, NY, 10151, US

(21) Appl. No.: 12/596,951

(22) PCT Filed: Apr. 28, 2008
(86) PCT No.: PCT/KR2008/002400

§ 371 Date: Oct. 21, 2009

(30) Foreign Application Priority Data
Apr. 30, 2007 (KR) 10-2007-0042059

(51) Int. Cl. A61K 8/97; A61Q 17/04

(52) U.S. Cl. 424/59

(57) ABSTRACT

The present invention relates to a composition for external preparation for UV protection comprising an extract of Rehmannia glutinosa (‘suk-ji-hwang’) as an active ingredient. Since the composition for external preparation for UV protection according to the present invention comprises an extract of Rehmannia glutinosa (‘suk-ji-hwang’) as an active ingredient, it has high skin safety and compatibility to the skin and shows high UV protection effect, and therefore, can be effectively used for preventing skin damage from daily exposure to UV radiation.

### TECHNICAL FIELD

The present invention relates to a composition for external preparation for protecting ultraviolet (UV) radiation which comprises an extract of Rehmannia glutinosa.

### BACKGROUND ART

Ultraviolet (UV) is indispensable to produce vitamin D in the human skin. However, it has been well-known in the art that when the skin is, overexposed to UV radiation, skin cancer, melasma, freckles or melanosis is occurred, and the generation of lipid peroxides and wrinkles is stimulated, leading to premature skin aging.

Therefore, many cosmetic manufactures have endeavored to protect the skin from various kinds of skin troubles that might be caused by overexposure to UV radiation and achieve skin whitening and anti-aging of the skin through the inhibition of lipid peroxide production by combining some quantity of UV screening agents with typical basic cosmetics as well as products for protecting UV radiation such as a sunscreen or a sunblock.

However, since most UV screening agents commonly used in the products for UV protection and the basic cosmetics have been reported as artificially synthesized products that are responsible for inducing skin safety, i.e., skin irritability, allergy or light toxicity, all countries of the world have regulated the content of UV screening agents used therein.

In order to overcome the skin safety problems of such artificially synthesized UV screening agents, extensive research on the development of natural UV screening agents having low skin troubles is actively underway in many countries. For instance, Japanese Patent Laid-Open Publication Nos. 86-87,610, 86-87,611, 86-87,612 and 86-87,609 disclose cosmetics comprising humic acids derived from natural coal; the United Kingdom Patent No. 2198042A discloses cosmetics comprising tyrosine derivatives; Germany Patent No. 3,513,928 discloses cosmetics comprising an extract from aloe. However, if a large amount of the above is applied to skin to improve UV protection due to their rather poor UV protection effect, it may result in skin problems in the applied area.

Meanwhile, Rehmannia is a perennial medicinal herb belonging to a kind of figwort, and Rehmannia's root is used medicinally in traditional oriental medicine. Raw roots are called ‘sang-ji-hwang’, dried roots are called dried ‘geon-ji-hwang’, and steamed roots are called ‘suk-ji-hwang’. ‘Suk-ji-hwang’ is used as a hematic and useful for the treatment of menstrual irregularity, infirmity, children ateliosis, dementia, premature ejaculation and impotence. ‘Sang-ji-hwang’ is useful for infirmity, hematemesis, epistaxis, metrorrhagia, menstrual irregularity and constipation, and ‘suk-ji-hwang’ is effective for thirst caused after febrile disease and by fever inside the organs and stops hematemesis and epistaxis.

‘Suk-ji-hwang’ has a sweet taste with bitterness. In addition, its warm properties strengthen the blood and replenish vitality (materials essential for originating and maintaining a life), which helps to treat a twinge of pain in one's waist and knees, menstrual irregularity, dizziness, hair blackening, etc.

‘Suk-ji-hwang’ is a major drug stuff of ‘samul-tang’, and used for treating ‘nae-yul’ (dry mouth caused by internal fever of body), throat xerosis, thirst and the like caused by infirmity among various kinds of chronic diseases. ‘Samul-tang’ has been widely used for treating over bleeding owing to women's birth and menstruation, infirmity, dizziness and the like. Further, ‘suk-ji-hwang’ has been taken by brewing a tea for treating ‘huh-dam’ from ancient times, and used in combination with Poria cocos and Pinellia tuber for cough and asthma.

### DISCLOSURE

### Technical Solution

The present inventors have therefore endeavored to develop a product by using natural substances with high skin safety and UV protection effect. As a result, they have found that an extract of Rehmannia glutinosa (‘suk-ji-hwang’), which is prepared by streaming and drying Rehmannia glutinosa, exhibits excellent UV protection effect, and completed the invention by applying the above discovery to a product.

Accordingly, the objective of the present invention is to provide a composition for external preparation for UV protection comprising an extract of Rehmannia glutinosa (‘suk-ji-hwang’) as an active ingredient.

### Advantageous Effects

Since the composition for external preparation comprising an extract of Rehmannia glutinosa (‘suk-ji-hwang’) according to the present invention shows high skin safety and compatibility to the skin and provides high UV protection effect, it can be effectively used for preventing skin damage from daily exposure to UV radiation.

### DESCRIPTION OF DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a graph illustrating UV protection rates of hot-water extracts from Rehmannia glutinosa (‘suk-ji-hwang’) prepared in Test Example 1.

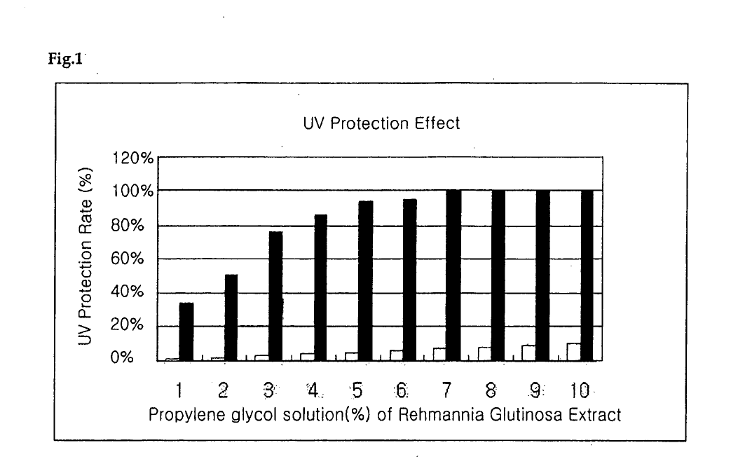

### DETAILED DESCRIPTION

The present invention is characterized by a composition for external preparation for UV protection comprising an extract of Rehmannia glutinosa (‘suk-ji-hwang’) as an active ingredient.

Hereinafter, the present invention will be explained in more detail.

The present invention relates to a composition for external preparation for

UV protection which is capable of preventing skin damage from daily exposure to UV radiation, wherein the composition comprises an extract of Rehmannia glutinosa (‘suk-ji-hwang’) as an active ingredient which shows excellent skin safety and compatibility to the skin and provides high UV protection effect.

The composition of the present invention comprises an extract of Rehmannia glutinosa (‘suk-ji-hwang’) as an active ingredient which is prepared by extracting Rehmannia glutinosa (‘suk-ji-hwang’) with water, alcohol according to a conventional solvent extraction method and purifying the same. At this time, the alcohol suitable for the present invention is exemplified by propylene glycol, ethanol and the like, but the use of ethanol is more preferable to achieve higher UV protection effect.

The Rehmannia glutinosa (‘suk-ji-hwang’) extract can be applied to a composition for external preparation such as cosmetics, ointments and the like. Further, basic ingredients of the cosmetics and ointments are commonly used in the art, and there is no limitation to the kinds of ingredients.

The Rehmannia glutinosa (‘suk-ji-hwang’) extract can be used as an ingredient for emulsion or dispersion products for cosmetics such as a cream, a liquid, a gel, a paste and the like, and applied to a toner, an astringent, an essence and the like to provide UV protection. In addition, it can be used for cosmetics such as a serum, a make-up base, a foundation, a powder, a concealer and the like.

The Rehmannia glutinosa (‘suk-ji-hwang’) extract may be used in the amount of 1 to 50 wt %, preferably 5 to 50 wt % based on the total weight of the composition for external preparation. If the amount of the Rehmannia glutinosa (‘suk-ji-hwang’) extract is too low, the composition may not be able to provide effective UV protection. In contrast, if the amount is too high, it may not be suitable for cosmetics due to a dark color peculiar to the Rehmannia glutinosa (‘suk-ji-hwang’) extract.

The following examples illustrate the invention but they should not be construed as limiting the scope of the instant invention.

### Test Example 1

After mixing 100 g of Rehmannia glutinosa (‘suk-ji-hwang’) with 900 g of distilled water, the mixture was boiled at 100° C. and filtered. The filtrate obtained above was re-boiled in a water bath, to obtain an extract of Rehmannia glutinosa. Thus obtained Rehmannia glutinosa (‘suk-ji-hwang’) hot-water extract was subjected to measure a UV protection rate by contents.

Namely, the Rehmannia glutinosa (‘suk-ji-hwang’) propylene glycol extract samples with different contents were thinly plated onto a slide glass for an optical microscope as a magic tape (3M, Magic Tape), and their UV protection rates were directly measured by using an ULTRAVIOLET DIGITAL METER (Quick). The UV protection rates measured above are shown in Table 1.

TABLE 1

| | Ingredient | | |
|---|---|---|---|
| | Rehmannia | | |
| | glutinosa (‘suk-ji- | | |
| | hwang’) extract | Propylene glycol | UV protection rate |
| | content (g) | content (g) | (%) |
| Sample 1 | 1 | 99 | 34 |
| Sample 2 | 2 | 98 | 50 |
| Sample 3 | 3 | 97 | 76 |
| Sample 4 | 4 | 96 | 86 |
| Sample 5 | 5 | 95 | 94 |
| Sample 6 | 6 | 94 | 95 |
| Sample 7 | 7 | 93 | 100 |
| Sample 8 | 8 | 92 | 100 |
| Sample 9 | 9 | 91 | 100 |
| Sample 10 | 10 | 90 | 100 |

As shown in Table 1, it was found that as the Rehmannia glutinosa (‘suk-ji-hwang’) extract's content is increased, the UV protection rate is proportionally improved. It was also possible to completely block UV radiation with only 7 wt % of the Rehmannia glutinosa (‘suk-ji-hwang’) extract. These results suggest that the Rehmannia glutinosa (—suk-ji-hwang’) extract is well dispersed in a solution, and thereby, exhibits superior UV protection effect at a low content.

### Test Example 2

A base composition for a cream was prepared by mixing 15 g of cetanol, 1 g of white wax, 10 g of propylene glycol, 2 g of sodium lauryl sulfate and 72 g of distilled water.

The base composition was mixed with the Rehmannia glutinosa (‘suk-ji-hwang’) hot-water extract obtained in Test Example 1 at different ratios as shown in Table 2, to prepare compositions for UV protection in a cream type.

The compositions for UV protection prepared by using the following samples 11 to 15 were thinly plated onto a slide glass for an optical microscope as a magic tape (3M, Magic Tape), and their UV protection rates were directly measured by using a ULTRAVIOLET DIGITAL METER (Quick). The UV protection rates measured above are shown in Table 2.

TABLE 2

| | Ingredient | | |
|---|---|---|---|
| | Rehmannia | | |
| | glutinosa (‘suk-ji- | | |
| | hwang’) extract | | UV protection rate |
| | content (g) | Base content (g) | (%) |
| Sample 11 | 1 | 99 | 20 |
| Sample 12 | 5 | 95 | 30 |
| Sample 13 | 20 | 80 | 70 |
| Sample 14 | 30 | 70 | 97 |
| Sample 15 | 40 | 60 | 100 |

As shown in Table 2, it was found that although the composition comprising the Rehmannia glutinosa (‘suk-ji-hwang’) extract as an active ingredient was mixed with the base composition in a cream type, it shows high UV protection effect. In case of using the Rehmannia glutinosa (‘suk-ji-hwang’) extract in the amount of 30 wt %, UV was almost completely protected. The above amount was greater than that of Test Example 1, but the composition still showed high UV protection activity.

### Formulation Example 1

### Preparation of a Skin

A skin comprising the Rehmannia glutinosa (‘suk-ji-hwang’) hot-water extract was prepared according to the following ingredients and contents by using a common method for preparing a skin in the art.

### [Ingredients]

Rehmannia glutinosa (‘suk-ji-hwang’) extract 3.0 wt %, polyoxyethylene hardened caster oil 0.5 wt %, glycine 3.0 wt %, dipotassium glycyrrhizate 0.1 wt %, 1,3-butylene glycol 3.0 wt %, sodium hyaluronate 0.1 wt %, ethanol 5.0 wt %, antioxidant 0.1 wt %, triethanolamine 0.1 wt %, EDTA 0.1 wt %, an adequate amount of a preservative, a residual amount of distilled water

### Formulation Example 2

### Preparation of a Cream

A nutrient cream comprising the Rehmannia glutinosa (‘suk-ji-hwang’) hot-water extract was prepared according to the following ingredients and contents by using a common method for preparing a nutrient cream in the art.

### [Ingredients]

Rehmannia glutinosa (‘suk-ji-hwang’) extract (3.0 wt %), 1,3-butylene glycol (3.0 wt %), glycerin (3.0 wt %), hydrogenated lecithin (1.0 wt %), octyldodecanol (3.0 wt %), trioctanoin (2.0 wt %), stearic acid (1.5 wt %), cetostearyl alcohol (2.0 wt %), polysorbate 60 (1.5 wt %), sorbitan sesquioleate (2.0 wt %), dimethicone (3.0 wt %), an antioxidant (0.3 wt %), zanthan gum (0.2 wt %), triethanolamine (0.1 wt %), EDTA (0.1 wt %), an adequate amount of a preservative, a residual amount of distilled water

### Formulation Example 3

### Preparation of an Essence

An essence comprising the Rehmannia glutinosa (‘suk-ji-hwang’) hot-water extract was prepared according to the following ingredients and contents by using a common method for preparing an essence in the art.

### [Ingredients]

Active ingredient (10.0 mg), glycerin (3.0 mg), EDTA (0.05 mg), benzophenone-9 (0.04 mg), carboxyvinyl polymer (0.2 mg), triethanolamine (0.18 mg), Octyldodeceth-25 (0.6 mg), glycerylmonostearate (1.0 mg), a preservative (0.01 mg), fragrance (0.01 mg), a residual amount of distilled water

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A composition for external preparation for UV protection comprising an extract of Rehmannia glutinosa (‘suk-ji-hwang’) as an active ingredient.

2. The composition for external preparation for UV protection according to claim 1, which comprises the extract of Rehmannia glutinosa (‘suk-ji-hwang’) in the amount of from 1 to 50 wt % based on the total weight of the composition.

3. A cosmetic product for UV protection comprising an extract of Rehmannia glutinosa (‘suk-ji-hwang’) as an active ingredient.

4. The cosmetic product according to claim 3, which comprises the extract of Rehmannia glutinosa (‘suk-ji-hwang’) in the amount of from 1 to 50 wt % based on the total weight of the cosmetic product.

5. The cosmetic product according to claim 3, which is prepared in the form of a cream, a liquid, a gel or a paste.

6. The cosmetic product according to claim 4, which is prepared in the form of a cream, a liquid, a gel or a paste.
